(19) United States
(12) Patent Application Publication
(10) Pub. No.: US 2010/0152509 A1
(43) Pub. Date: Jun. 17, 2010

(54) Fuel Produced From a Biomass

(75) Inventor: Rune Ekman, Tagarp (SE)

Correspondence Address: SMITH, GAMBRELL & RUSSELL, 1130 CONNECTICUT AVENUE, N.W., SUITE 1130, WASHINGTON, DC, 20036, US

(73) Assignee: Reac Fuel AB, Lundstate (SE)

(21) Appl. No.: 12/532,882

(22) PCT Filed: Mar. 25, 2008
(86) PCT No.: PCT/SE08/00217

§ 371 Date: Feb. 10, 2010

Related U.S. Application Data
(60) Provisional application No. 60/907,336, filed on Mar. 29, 2007.

(30) Foreign Application Priority Data
Mar. 29, 2007 (SE) 0700840-2

(51) Int. Cl. C07C 1/00

(52) U.S. Cl. 585/324; 585/310

(57) ABSTRACT

The present invention relates to a method of production of a fuel containing at least one hydrocarbon from a biomass, comprising the steps of: -A) chemical decomposition of the biomass in a solvent to at least one chemical intermediate compound chosen from the group consisting of a hexose, a pentose and a derivative of lignin, or a combination thereof, optionally produced together with at least one other target chemical, to form a first solution; - B) complete reduction of the first solution with a reducing agent to at least one hydrocarbon to form a fuel; wherein the steps A) and B) are performed below 300° C.

### TECHNICAL FIELD

The present invention relates to a method of production of a fuel containing at least one hydrocarbon, from a biomass.

### BACKGROUND ART

The interest for biofuels is rapidly increasing over the world. This is of course due to environmental aspects as well as other problems with a high dependency on fuels from crude oil, such as economic aspects, for instance a fluctuating but over time increasing spot price of the crude oil world wide.

Biofuels are fuels produced from living organisms (biomass). In contrast fossil fuels have been outside the natural cycle of operation on earth for millions of years, which causes known problems. In contrast to the combustion of fossil fuels, biofuels in principle do not contribute to the net accumulation of carbon dioxide in the atmosphere. The amount of carbon dioxide formed at combustion is the same amount absorbed by the plant during growth. One definition of a biofuel is any fuel with an 80% minimum content by volume of materials derived from living organisms harvested within the ten years preceding its manufacture.

Different types of refined biofuels are e.g. biogas and biodiesel, but other examples are (bio)ethanol and (bio)methanol. Biodiesel typically refers to a diesel equivalent, processed fuel derived from biological sources, such as e.g. vegetable oils, which can be used in unmodified diesel engined vehicles. It is thus distinguished from the straight vegetable oils (SVO) or waste vegetable oils (WVO) used as fuels in some modified diesel vehicles. According to one aspect, the term biodiesel refers to alkyl esters made from the transesterification of vegetable oils or animal fats. Biodiesel is biodegradable and non-toxic, and typically produces about 60% less net emissions of carbon dioxide in comparison to diesels based on petroleum (mineral oil).

Biogas typically refers to a (biofuel) gas produced by the anaerobic digestion or fermentation of organic matter. Biogas is comprised primarily of methane and carbon dioxide. As a fuel for vehicles, biogas is environmentally advantageous in comparison to gasoline and diesel, but as well in comparison to E85 (85 wt % ethanol/15 wt % gasoline) and natural gas.

Ethanol fuel is an alternative to gasoline. It can be combined with gasoline in any concentration up to pure ethanol (E100). Ethanol can be produced by fermentation of sugar or by hydration of ethylene from petroleum and other sources. Currently, there is an interest in ethanol production from crops, that is to what is referred to as bioethanol. It is readily obtained from the starch or sugar in a wide variety of crops.

Common for all biofuels is the fact that they are produced from biomass, of which starch and cellulose are the two most important components. Starch is a complex carbohydrate which is soluble in water and it is used by species plants as a strategic way to store excess glucose. Starch consists of molecular chains of glucose which can be straight (amylose) or branched (amylopectine). Starch is in other words a polysaccharide of alfa-glucose. Cellulose on the other hand is a long-chain polysaccharide of beta-glucose.

It is known how to convert biomass to biofuels by pyrolysis, which is the chemical decomposition of organic materials by heating in the absence of oxygen or any other reagents, except possible steam. Pyrolysis is a special case of thermolysis. Fast pyrolysis of biomass feedstocks is required to achieve high yields of liquids. It is characterized by rapid heating of the biomass particles and a short residence time of product vapors (0.5 to 2 s). Rapid heating means that the biomass must be ground into fine particles and that the insulating char layer that forms at the surface of the reacting particles must be continuously removed. At low temperature pyrolysis the material is heated to a temperature below 500° C. High temperature pyrolysis is consequently performed above 500° C.

There are different possible products achievable by pyrolysis of biomass. At pyrolysis of cellulose, one gaseous, one liquid and one solid fraction is formed. The gaseous fraction consists of combustible gases, such as hydrogen gas and methane. Pyrolytic oil together with a water fraction, e.g. containing methanol and acetic acid, are possible components of the liquid fraction. The solid fraction looks like char coal, but one should understand that the greater part of the inorganic material of raw material will be comprised in this fraction. The yield at pyrolysis depends on many different factors, e.g. the composition of the raw material, temperature and pressure.

Other known conversion types of biomass to fuels are e.g. catalytic reduction, in which organic material is mixed with carbon monoxide at a pressure of about 300 atm and a temperature of 350-400° C. One advantage of this process in comparison to pyrolysis is the fact that there is more or less only one product formed. On the other hand this process is often more expensive than pyrolysis.

Yet another reaction type is methane fermentation, which is a biochemical process which is possible to use on biomass material not containing large amounts of lignin. Suitable start material are e.g. agriculture waste or waste from people. The methane fermentation is performed under anaerobic conditions with influence of bacteria.

There are different problems with these different known biofuel types. One problem is e.g. a low intrinsic energy density of some of the biofuels. Ethanol is one example of such a biofuel due to the fact that it contains a high level of oxygen and not just carbon and hydrogen atoms. Another problem with many of the biofuels is a low energy yield with reference to the start material in comparison to the produced biofuel. For example, a resulting biogas only has a conversion efficiency of about 60% of the carbon inlet in a solid biomass. Moreover, high losses during the production is also a common problem, which of course affects the economy of the production negatively. Some other biofuels are in themselves very expensive to produce due to expensive start materials in comparison, expensive process equipment etc.

The object of the present invention is to provide a process for the production of a biofuel which solves the problems described above, i.e. which has economic advantages in relation to yield and process costs but at the same time produces biofuels having high intrinsic energy levels, i.e. only having a high hydrogen carbon content and not any oxygen. Moreover, inter alia the values of the estimated conversion efficiency, energy value in the final biofuel and produced fuel energy in relation to the field area are kept high for biofuels produced according to the process of the present invention in comparison to biofuels produced by known processes for production of biofuels (see table 1 in the conclusions).

### SUMMARY OF THE INVENTION

The object described above is solved by the method of production of a fuel containing at least one hydrocarbon from a biomass according to the present invention, which process comprises the steps of:

A) chemical decomposition of the biomass in a solvent to at least one chemical intermediate compound chosen from the group consisting of a hexose, a pentose and a derivative of lignin, or a combination thereof, optionally produced together with at least one other target chemical, to form a first solution;

B) complete reduction of the first solution with a reducing agent to at least one hydrocarbon to form a fuel;

wherein the steps A) and B) are performed below 300° C.

With chemical decomposition is meant cleavage of longer hydrogen carbon chains to the specified chemicals above. This decomposition could also be specified as a dissolution together with a monomerisation.

The product of course possible from the reduction comprises different hydrocarbon components, and additionally possible other product components of which some are preferably removed from the fuel. With reduction is in this case meant the removal of oxygen from the first solution. With completo reduction is meant a reduction where there is substantially no oxygen left bound in the target products in the first solution from step A).

As notable from above, the process according to the present invention does not include a pyrolysis step. It is in fact very surprising that only the two steps of chemical decomposition and complete reduction are possible to utilise for the production of a fuel (biofuel) from a biomass.

### DETAILED DESCRIPTION OF THE INVENTION

Below, the present invention will be described in more detail with reference to some specific embodiments. The explanation is provided to enhance the understanding of these specific embodiments, and these embodiments and the explanation thereof should not be interpreted as a limitation of the present invention. The scope of the present invention is defined by the appended claims.

There are different possible main intermediate compounds formed in step A), which of course is dependent on the source of the biomass, but as well other conditions of the chemical decomposition. As mentioned above, the possible chemical intermediate compounds are hexoses, pentoses and derivatives of lignin, or combinations thereof, optionally together with other chemicals or product components.

A hexose is a monosaccaride with 6 carbon atoms having the chemical formula of C₆H₁₂O₆. Hexoses are classified by functional groups, where aldohexoses have an aldehyde at position 1, and ketohexoses have a ketone at position 2. The aldohexoses have 4 chiral centres for a total of 16 possible aldohexose stereoisomers. The D- or L-configuration of aldohexoses is based on the orientation of the hydroxyl group at position 5. The eight D-aldohexoses are D-allose, D-altrose, D-glucose, D-mannose, D-gulose, D-idose, D-galactose, and D-talose. Of these D isomers all, except altrose, are said to occur naturally. No L-isomers are, however, found naturally. According to the present invention, all of the above are possible intermediate product components from step A), but glucose is however the most likely. The ketohexoses have 3 chiral centres and therefore 8 possible stereoisomers. Of these, only the four D-isomers are known to occur naturally, which are D-psicose, D-fructose, D-sorbose and D-tagatose. The ketohexoses are also possible intermediate product components from step A) according to the present invention, dependent of course on the source of biomass used.

A pentose on the other hand is a monosaccharide with 5 carbon atoms. They either have an aldehyde as the functional group in position 1 (aldopentoses), or a ketone in position 2 (ketopentoses). The aldopentoses have 3 chiral centres and therefore 8 different stereoisomers. The four D-aldopentoses are D-ribose, D-arabinose, D-xylose, and D-lyxose. The ketopentose on the other hand have 2 chiral centres and therefore 4 possible stereo-isomers, which are ribulose (L- and D-form) and xylulose (L- and D-form). The pentoses are also possible intermediate product components from step A).

Lignin, which is sometimes called “lignen” is a compound that is most commonly derived from wood and is an integral part of the cell walls of plants. Lignin is a 3-D polymer and a condensed product derived from phenolic acids like ferulic acid, benzoic acid, coumaric acid and cinnamic acid. The carbon weight is up to 50% higher than in carbohydrates. It is one of most abundant organic compounds on earth after cellulose and chitin. Lignin makes up about one-quarter to one-third of the dry mass of wood. It has several unusual properties for being a biopolymer, such as having a network structure and lacking a defined primary structure. As mentioned above, possible intermediate product components from step A) according to the present invention are derivative of lignin, which in this case should be interpreted as building stones making up the entire lignin molecule, that is decomposition derivatives of lignin. Below, the structure of a small piece of a lignin polymer is illustrated:

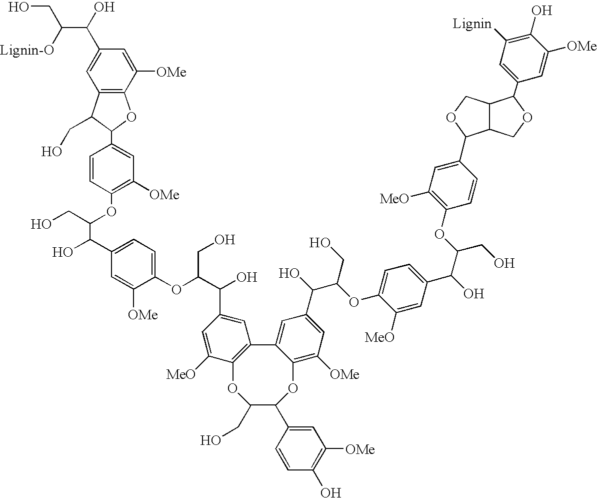

Intermediate decomposition product components of lignin in step A) according to the present invention are normally alcohol derivatives from the splitting of a lignin molecule. Below, there is provided the structures of the three commonly occurring monolignols:

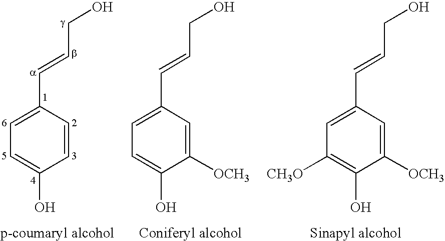

There are in other words three main types of monolignols, namely paracoumaryl alcohol, coniferyl alcohol and sinapyl alcohol. Consequently, according to one specific embodiment of the present invention, the at least one chemical intermediate compound of step A) is at least one of p-coumaryl alcohol, coniferyl alcohol, sinapyl alcohol, or a combination thereof. There are, however, a number of other monolignols present in special plants or in low concentration in common plants. Different plants use different monolignols. These ones are also possible intermediate product components from step A) according to the present invention.

As mentioned above, glucose is an important component of starch. Maltose is a disaccharide formed from two units of glucose. The addition of another glucose unit yields maltotriose, and a chain with four glucose is maltotetrose, etc. Further additions will produce dextrins, also called maltodextrins, and eventually starch will be the result.

Cellobiose on the other hand is a disaccaride subunit of cellulose composed of two glucose molecules linked in a β(1→4) bond. Cellobiose is e.g. an indispensable part of fabric, especially cotton, jute and other cellulosic materials. Cellobiose is also an essential part of all plant structures. Its strong —OH bond interactions with neighbouring cellobiose molecules contributes greatly to the plant's structural resilience.

Therefore, according to one specific embodiment, the at least one chemical intermediate compound of step A) is at least one of glucose, maltose, cellobiose, or a combination thereof. These ones are probable as intermediate decomposition products from step A) according to the present invention.

As discussed in some extent above, different types of biomass have different compositions and possible contain different components. There are, however, some important ones which are frequent occurring, such as e.g. cellulose and/or starch. According to one specific embodiment of the present invention, the biomass utilised in the method of production of a fuel comprises starch, cellulose, hemicellulose, lignin, lignocellulose or a combination thereof.

Cellulose and starch have been discussed above, but below some of the other important possible components of a biomass will de described. Hemicellulose contains many different sugar monomers. In contrast, cellulose contains only anhydrous glucose. For instance, besides glucose, sugar monomers in hemicellulose can e.g. include xylose, mannose, galactose, rhamnose, and arabinose. Hemicelluloses contain most of the D-pentose sugars and occasionally small amounts of L-sugars as well. Xylose is always the sugar monomer present in the largest amount, but mannuronic acid and galacturonic acid also tend to be present. These are also possible intermediate decomposition products from step A).

Moreover, lignocellulosic biomass is biomass composed primarily by cellulose and lignin. Examples of lignocellulosic biomass are all types of trees, grasses, and agricultural residues.

One interesting aspect with reference to the crude material used for step A) of the method of production according to the present invention, is the fact that even if the main start material will be a biomass, it may be possible to utilise some other residues additionally, such as plastic materials. As long as the residues as well are hydrocarbon containing materials, they are possible to convert into hydrocarbon chemicals having interesting fuel properties. This is of interest in the case where e.g. a farmer stores or keeps the biomass material in e.g. plastic containers or bags, due to that these together with their biomass content may be used as the start material for the chemical decomposition step A) of the method according to the present invention.

There are of course different products possible to achieve with the process or method of production according to the present invention. In this sense, the composition of the biomass as well as the conditions used are of importance. According to one specific embodiment of the present invention, the at least one hydrocarbon produced in step B) is at least one of an alkane or alkene, or a combination thereof.

One parameter important to take into account is the dry matter content level of the biomass used in the process according to the present invention. According to one specific embodiment of the present invention, the biomass has a dry matter content level of at least 85 wt %, preferably at least 95 wt %. With reference to this aspect, there are of course different levels of the natural dry matter content level of different types of biomass in comparison to each other. As an example, typical bark chips have a dry matter content as low as e.g. 45 wt %. One has to understand that it is of course possible to dry some of the types of biomass having a lower level of dry matter content, if necessary, before the chemical decomposition step. There are, however, types of biomass suitable without the use of a predrying step. Hemp, for instance, is an energy-intensive crop, which after full annual growth by a harvesting waiting strategy, i.e. without industrial drying, has a field dry matter content level of about 90 wt %, and sometimes even above 95 wt %. This means that hemp is a crop which is harvested in dry form and is a high-yield crop. Therefore, according to one specific embodiment of the present invention, the biomass is a hemp (or an industrial hemp) or a biomass mixture comprising hemp.

Another important level in relation to the dry level of different biomasses is a dry matter content level of at least 87% due to the fact that this is known to be the lower limit at which fungi can not grow or “attack” wood materials or biomass materials. This is of course of interest for the storage of biomasses.

Another factor of interest is the growing of different types of plants. For instance, a plant that usually germinates, flowers and dies in one year is what is often referred to as an annual plant. True annuals will only live longer than a year if they are prevented from setting seed. Some seedless plants can also be considered annuals even though they do not flower. One seed-to-seed life cycle for an annual can occur in as little as a month in some species, though most last several months. E.g. oilseed rape can go from seed-to-seed in about five weeks under a bank of fluorescent lamps in a school classroom. Many desert annuals are termed ephemerals because their seed-to-seed life cycle is only a few weeks. They spend most of the year as seeds to survive dry conditions. Examples of true annuals include corn, lettuce, pea, cauliflower, and bean. The annual plants is of course of interest because they could be considered to be “self drying” plants. Therefore, according to one specific embodiment of the present invention, the biomass comprises at least one type of annual plant. Hemp is as mentioned above an annual plant possible to use according to the present invention. Another example possible to use is cereal straw.

The chemical decomposition step A) may be performed in different ways and with different chemicals taking part. According to one specific embodiment of the present invention, step A) is performed by chemical decomposition of the biomass in a solvent chosen from the group consisting of supercritical water (SCW), a superacid, hydrazine, ammonia and an amine, or a combination thereof. The amines can both have usual chains or be cyclic. One example of a cyclic one is aniline.

A superacid is an acid with an acidity greater than that of 100% sulphuric acid. A supercritical fluid is any substance at a temperature and pressure above its thermodynamic critical point. It has the unique ability to diffuse through solids like a gas, and dissolve materials like a liquid. Additionally, it can readily change in density upon minor changes in temperature or pressure.

According to one specific embodiment of the present invention, step A) is performed by chemical decomposition of the biomass in a solvent chosen from the group consisting of dry and pressurised ammonia, supercritical ammonia, a dry and pressurised amine, and a dry and pressurised hydrazine. Just like the high dry matter content of the biomass, it may be of importance according to one specific embodiment of the present invention to use a dry and pressurised nitrogen containing solvent. In this sense it is important to understand that it is not only supercritical water that is of interest, but e.g. supercritical ammonia is also of interest to use.

It is possible to perform the reduction step b) of the present invention by utilising different chemicals, in this case reducing agents, normally strong such reducing agents. According to one specific embodiment of the present invention, the reduction step B) is performed by reacting the first solution with a reducing agent chosen from the group consisting of hydrogen sulphide, white phosphorous, hydrazine, and a hydride containing silane, that is a silane having at least one H⁻ group (hydride ion) bounded thereto. A hydride ion is a negatively charged hydrogen ion, that is an ion that has two electrons in its electron shell.

The hydride containing silane compounds according to the invention has the general formula of:

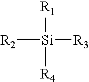

in which at least one of R₁ to R₄ is a H⁻ group. Normally at least two of R₁ to R₄ are H⁻ groups. Examples of silanes according to the invention are diethyl silane:

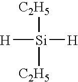

or n-buthyl-silane:

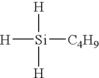

but there are of course other ones possible as well.

In this sense the high dry matter content level is also of interest due to the fact that silanes are not selective reducing agents and normally react with all available oxygen, both free and bounded. Therefore it is normally of interest to keep the water content and hence available oxygen content in the water molecules as low as possible, when using silanes as reducing agents.

According to another specific embodiment of the present invention, step A) is performed by chemical decomposition of the biomass in dry and pressurised ammonia or supercritical ammonia and step B) is performed by reacting the first solution with a reducing agent which is Na, K, Ca in dissolved and/or melted form or NaH, KH and CaH₂, i.e. the corresponding metal hydrides. The alkali metals electrons are in this case solved in e.g. ammonia.

Even though glucose is the important main target chemical produced in step A) according to the method of the present invention, other target chemicals may also be produced. Therefore, according to one specific embodiment of the present invention, at least one phenol or other alcohol, having from 6 to 20 carbon atoms, is produced in step A) as the optional target chemical.

Process parameters, such as pressure and temperature, vary and are optimised dependent on the different chemicals taking any part in step A) and B). According to one specific embodiment of the present invention, the average temperature for performance of steps A) and B) are below 220° C., preferably below 190° C.

Regeneration of important chemicals, such as e.g. solvents, reducing agents and/or energy carriers, as well as the removal of undesirable by-products may be of importance in the method of production according to the present invention. Regeneration may be performed according to known and classical techniques according to state of the art. Moreover, according to one specific embodiment, undesirable substances are removed before or in the steps A) and/or B). According to another specific embodiment, the undesirable substances being removed before or in step A) comprise molecules containing N and/or S. N and S containing substances are not desirable by-products because they produce nitric acid and sulphuric acid, respectively, when combusted. Some amounts of SOₓ and NOₓ gases are also possibly produced. Other possible chemicals being removed are e.g. proteins and metals.

The product from reduction step B), i.e. the raw fuel before the possible distillation (step C), may contain different chemicals, such as different alkanes having different chain lengths. According to one specific embodiment, however, the at least one hydrocarbon produced in step B) are at least one of an alkane or alkene having 8 carbon atoms.

Due to the fact that hydrocarbon chains having 8 carbon atoms, such as an octane, often are of great interest in the fuel industry, these are of significance here as well. According to one specific embodiment of the present invention, the fuel being produced in step B) comprises at least 90 wt %, preferably at least 95 wt %, of at least one alkane having 8 carbon atoms.

As hinted above, a possible step C) consisting of distillation is also possible in the method according to the present invention. Therefore, according to one specific embodiment, the method according to the present invention additionally comprises another step which is:

C) fractionated distillation of the fuel produced in step B).

The distillation step C) may be of a type normally used for distillation. Exactly how the distillation is performed, the equipment used therefore and specific conditions like temperature and pressure depend on many factors, e.g. the desired end product and requirements thereof but of course as well the raw biofuel mixture fed to the distillation.

The fuel or biofuel produced according to the method of the present invention may be of different type, and according to one specific embodiment, the fuel being fractionated out from step C) is chosen from the group of bio equivalents to diesel, an Otto fuel and a jet fuel, or a combination thereof, which are equivalents with reference to a combustion point of view. With equivalents with reference to a combustion point of view is here meant that the fractionated biofuel according to the present invention works in the same way as a corresponding non-biofuel in relation to combustion effect, thermal value or energy value, composition of combustion products, etc. One important factor, when discussing classical fuels, is the octane content and octane number. According to one specific embodiment of the present invention, the fuel being fractionated out from step C) comprises at least 95 wt % of 2,2,4-trimethylpentane (an octane value of at least 95). As hinted, the molecule of 2,2,4-trimethylpentane has an octane value of 100.

The present invention also provides hydrocarbon containing fuels obtainable by the method of production according to the present invention.

### CONCLUSION

In table 1 below a biofuel produced according to the present invention is compared with other biofuels, namely biogas, ethanol and rapeseed methyl ester. As can be seen from table 1, when comparing the important parameters of transferred percentage energy from field, estimated conversion efficiency, energy value in final fuel, produced fuel energy, percentage energy yield and percentage needed field area, it is clear that the biofuel produced according to the process of the present invention has much better values than these other bio-alternatives.

TABLE 1
Comparison of a produced biofuel according to the present invention with other biofuels.

| | Transferred | | | | Percentage | Percentage |
|---|---|---|---|---|---|---|
| | percentage | Estimated | Energy | Produced | energy yield | needed |
| | energy from | conversion | value in | fuel energy | of (dry | of field |
| Fuel type | field¹ | efficiency | final fuel | (MWh/(ha*year)) | matter/(ha*year)) | area² |
| Produced | 96% dry | 93-99% | 11.9 kWh/kg | 56-60 | 93-99% | 1 |
| fuel accord- | matter = 58 | | liquid fuel or | | | |
| ing to the | MWh/(ha*year), | | 9.9 kWh/l | | | |
| present | harvested at the | | | | | |
| invention | lowest water | | | | | |
| | content and ash | | | | | |
| | content. | | | | | |
| Biogas | 96% dry | 60% | Gaseous | 36 | ~60% | 1.5-1.7 |
| | matter = 58 | | fuel | A gas has | | |
| | MWh/(ha*year), | | | 1/1000 in | | |
| | harvested as dry | | | energy density | | |
| | matter substance | | | in comparison | | |
| | and at high water | | | with a liquid. | | |
| | content and ash | | | Has to be | | |
| | content. | | | pressurised. | | |
| Ethanol | 48% dry | 336 l ethanol/ton | 8.2 kWh/kg | 14.2 | 24% | 3.9-4.1 |
| (density = | matter = 29 | wheat | or 6.5 kWh/l | | | |
| 0.79 g/cm³) | MWh/(ha*year), | | | | | |
| | harvested as | | | | | |
| | seed at low dry | | | | | |
| | matter and at | | | | | |
| | high ash content. | | | | | |
| Rapeseed | 48% dry | ~60% of the glu- | ~15 kWh/kg | 11.3 | 19% when | 4.9-5.2 |
| methyl | matter = 29 | cose equivalent | liquid fuel or | | crushing on a | 3.3-3.5 |
| ester | MWh/(ha*year), | E-content in the | 12 kWh/l. | | farm. | |
| | crushed out of | seed is in the | Some | | 28% at | |
| | fat rich seed. | rapeseed oil. At | variation | | chemical | |
| | | farm crushing | dependent | | extraction on a | |
| | | ⅔ of this is re- | on the | | process plant. | |
| | | covered, at fac- | average | | | |
| | | tory extraction | length of the | | | |
| | | 99%. | fatty acids in | | | |
| | | | the seed | | | |
| | | | (C6-C20). | | | |

¹Transferred percentage energy from the field of the total biomass above earth, if assumption is made that all crops give 12 tons dry matter substance recalculated to glucose equivalents.
²The amount of additional field area needed in comparison to use of the biofuel and its logistics according to the present invention, to cover the need of the Swedish transport sector, expressed relatively to the fuel and its logistics according to the present invention.
Index 1 corresponds to about 2 Mha of the Swedish total of 45 Mha.

1-24. (canceled)

25. A method of production of a fuel containing at least one hydrocarbon from a biomass, comprising the steps of:
A) chemical decomposition of the biomass in a solvent chosen from the group consisting of supercritical water (SCW), a superacid, hydrazine, ammonia and an amine, or a combination thereof, to at least one chemical intermediate compound chosen from the group consisting of a hexose, a pentose and a derivative of lignin, or a combination thereof, optionally produced together with at least one other target chemical, to form a first solution;
B) complete reduction of the first solution with a reducing agent chosen from the group consisting of hydrogen sulphide, white phosphorous, hydrazine, and a hydride containing silane, to at least one hydrocarbon to form a fuel.

26. The method of production of a fuel according to claim 25, wherein step A) is performed by chemical decomposition of the biomass in a solvent chosen from the group consisting of dry and pressurised ammonia, supercritical ammonia, a dry and pressurised amine, and a dry and pressurised hydrazine.

27. The method of production of a fuel according to claim 25 or 26, wherein the hydride containing silane is diethyl silane or n-buthyl-silane.

28. A method of production of a fuel containing at least one hydrocarbon from a biomass, comprising the steps of:
A) chemical decomposition of the biomass in dry and pressurised ammonia or supercritical ammonia, to at least one chemical intermediate compound chosen from the group consisting of a hexose, a pentose and a derivative of lignin, or a combination thereof, optionally produced together with at least one other target chemical, to form a first solution;
B) complete reduction of the first solution with a reducing agent, which is Na, K, Ca in dissolved and/or melted form or NaH, KH or CaH₂, to at least one hydrocarbon to form a fuel.

29. The method of production according to claim 25, wherein the steps A) and B) are performed below 300° C.

30. The method of production of a fuel according to claim 25, wherein the at least one chemical intermediate compound of step A) is at least one of glucose, maltose, cellobiose, or a combination thereof.

31. The method of production of a fuel according to claim 25, wherein the at least one chemical intermediate compound of step A) is at least one of p-coumaryl alcohol, coniferyl alcohol, sinapyl alcohol, or a combination thereof.

32. The method of production of a fuel according to claim 25, wherein the biomass comprises starch, cellulose, hemicellulose, lignin, lignocellulose or a combination thereof.

33. The method of production of a fuel according to claim 25, wherein the at least one hydrocarbon produced in step B) is at least one of an alkane or alkene, or a combination thereof.

34. The method of production of a fuel according to claim 25, wherein the biomass has a dry matter content of at least 85 wt %.

35. The method of production of a fuel according to claim 25, wherein the biomass has a dry matter content of at least 95 wt %.

36. The method of production of a fuel according to claim 25, wherein the biomass comprises at least one type of annual plant.

37. The method of production of a fuel according to claim 25, wherein the biomass is a hemp or a biomass mixture comprising hemp.

38. The method of production of a fuel according to claim 25, wherein at least one phenol or other alcohol, having from 6 to 20 carbon atoms, is produced in step A) as the optional target chemical.

39. The method of production of a fuel according to claim 25, wherein the average temperature for performance of steps A) and B) is below 220° C.

40. The method of production of a fuel according to claim 25, wherein the average temperature for the performance of steps A) and B) is below 190° C.

41. The method of production of a fuel according to claim 25, wherein undesirable substances are removed before or in the steps A) and/or B).

42. The method of production of a fuel according to claim 25, wherein undesirable substances are removed before or in the step A), the undesirable substances comprising molecules containing N and/or S.

43. The method of production of a fuel according to claim 25, wherein the at least one hydrocarbon produced in step B) are at least one of an alkane or alkene having 8 carbon atoms.

44. The method of production of a fuel according to claim 25, wherein the fuel being produced in step B) comprises at least 90 wt % of at least one alkane having 8 carbon atoms.

45. The method of production of a fuel according to claim 25, wherein the fuel being produced in step B) comprises at least 95 wt % of at least one alkane having 8 carbon atoms.

46. The method of production of a fuel according to claim 25, wherein the method additionally comprises another step which is:
C) fractionated distillation of the fuel produced in step B).

47. The method of production of a fuel according to claim 25, wherein the method additionally comprises another step which is:
C) fractionated distillation of the fuel produced in step B), and wherein the fuel being fractionated out from step C) is chosen from the group of bio equivalents to diesel, an Otto fuel and a jet fuel, or a combination thereof, which are equivalents with reference to a combustion point of view.

48. The method of production of a fuel according to claim 25, wherein the method additionally comprises another step which is:
C) fractionated distillation of the fuel produced in step B), and wherein the fuel being fractionated out from step C) comprises at least 95 wt % of 2,2,4-trimethylpentane.
